(19) United States
(12) Patent Application Publication
(10) Pub. No.: US 2015/0329515 A1
(43) Pub. Date: Nov. 19, 2015

(54) COMPOSITION AND METHOD FOR TREATING OR PREVENTING INFLUENZA VIRUS INFECTION

(71) Applicant: National Defense Medical Center, Taipei (TW)

(72) Inventors: An-Rong LEE, Taipei City (TW); Shu-Ting CHUNG, Taichung City (TW); Wen-Hsin HUANG, New Taipei City (TW)

(21) Appl. No.: 14/276,219

(22) Filed: May 13, 2014

(51) Int. Cl. C07D 311/30; C07C 49/84

(52) U.S. Cl. CPC C07D 311/30; C07C 49/84

(57) ABSTRACT

The present invention provides a pharmaceutical composition and method for treating or preventing influenza virus infection in a subject comprising administering the subject with a pharmaceutical composition comprising a pharmaceutically acceptable carrier and a therapeutically effective amount of compounds provided in this invention. In addition, the prevent invention provides new compounds for treating or preventing influenza virus infection.

### FIELD OF THE INVENTION

The present invention relates to a composition and method for treating or preventing influenza virus infection.

### BACKGROUND OF THE INVENTION

Influenza viruses are the most significant source of viral respiratory infections in humans worldwide, causing recurrent epidemics and global pandemics that bring about severe morbidity and mortality involving millions of people annually (De Clercq E., Nature Rev. Drug Discovery, 5, 1015-1025 (2006)). Influenza virus is an RNA virus of the Orthomyxoviridae family and can be classified into three types: A, B, and C (Lambert et al., N. Engl. J. Med., 363, 2036-2044 (2010)). Unlike influenza B and C viruses, which mainly infect humans, influenza A viruses infect a wide range of hosts, including humans, swine, birds, horses and whales (Medina et al., Nat. Rev. Microbiol., 9, 590-603 (2011)). H1N1 and H3N2 influenza A viruses have co-circulated since 1977. In the spring of 2009, a new virulent pandemic strain with the H1N1 antigenic subtype appeared and spread globally (Monsalvo et al., Nat. Med., 17, 195-199 (2011)).

There have been several antiviral agents approved to treat and prevent influenza virus infection. Amantadine and Rimantadine remain important in this regard. However, the rapid development of resistance and the serious toxic effects in the autonomic nervous system limited these two agents impractical in clinical use of antiviral efficacy. Two major specific neuraminidase (NA) inhibitors, oseltamivir (Tamiflu) and zanamivir (Relenza), have been successfully prepared by computer-aided drug design and extensively used for treatment of influenza virus infection. (Neumann et al., Nature, 459, 931-939 (2009)). The structures of NA inhibitors, in many cases, consist of a transition-state configuration. Therefore, the NA inhibitors perform their antiviral activity by targeting neuraminidase and thus effectively abolish the proliferation and spreading of viruses. The genesis of oseltamivir and zanamivir represent a new generation of antiviral agents associated with excellent efficacy and specificity. Unfortunately, these two potent NA inhibitors have adverse effects (Fuyuno I., Nature, 446, 358-359 (2007)). Moreover, recent studies have indicated that pandemic H1N1 Tamiflu-resistant (H1N1 TR) viral isolates have been progressively rising, which may pose the risk for increasing fatality in human beings (Moscona A., N. Engl. J. Med., 360, 953-956 (2009), and Gooskens et al., J. Am. Chem. Soc., 301, 1042-1046 (2009)). Therefore, the development of new therapeutic agents which have the potential for treating or preventing influenza virus infection is urgently needed.

### BRIEF SUMMARY OF THE INVENTION

It is unexpectedly found in the invention that some new compounds are effective in treating or preventing influenza virus infection, particularly Tamiflu-resistant influenza virus.

In one aspect, the invention provides a compound having the structure of Formula I or a pharmaceutically acceptable salt or a physiologically functional derivative thereof:

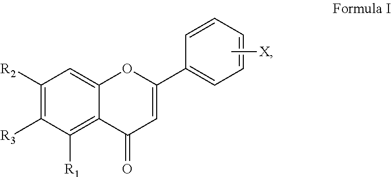

wherein X is F, Cl, Br, I, COOH, phenyl, NO₂;
each of R₁, R₂ and R₃ is OH, OCH₃ or O(CH₂)ₘ—CH₃, m is an integer of 1-3.

In one embodiment of the invention, wherein X is Cl or Br.

In another embodiment, wherein X is Br.

In other embodiments of the invention, wherein X is 2′-Cl, 3′-Cl, 4′-Cl, 2′,4′-(Cl)₂, 3′,4′-(Cl)₂, 3′-Br and 4′-F, 4′-COOH, 4′-phenyl, 4′-NO₂, 2′-Br, 3′-Br, 4′-Br.

In one embodiment of the invention, wherein each of R₁, R₂ and R₃ is OH or OCH₃.

In another embodiment, wherein X is 3′-Br, 4′-Br, and R₁═R₂═OH, R₃═OH or OCH₃.

In a yet aspect, the invention provides new compounds, which is selected from the group consisting of:

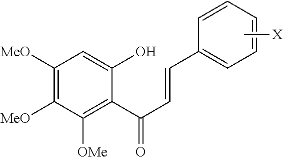

wherein X is H, 2′-Cl, 3′-Cl, 4′-Cl, 2′,4′-(Cl)₂, 3′,4′-(Cl)₂, 3′-Br and 4′-F, 4′-COOH, 4′-phenyl, 4′-NO₂, 2′-Br, 3′-Br, 4′-Br.

In a further aspect, the invention provides a pharmaceutical composition for treating or preventing influenza virus infection comprising the compound having the structure of Formula I or a pharmaceutically acceptable salt or a physiologically functional derivative thereof.

In one aspect, the invention provides a method for treating or preventing influenza virus infection in a subject comprising administering the subject with a pharmaceutical composition comprising a pharmaceutically acceptable carrier and a therapeutically effective amount of a compound having the general Formula I:

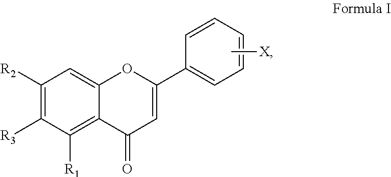

wherein X is F, Cl, Br, I, COOH, phenyl, NO₂;
each of R₁, R₂ and R₃ is OH, OCH₃ or O(CH₂)m-CH₃, m is an integer of 1-3.

In one embodiment of the invention, wherein X is Cl or Br.

In another embodiment, wherein X is Br.

In other embodiments of the invention, wherein X is 2′-Cl, 3′-Cl, 4′-Cl, 2′,4′-(Cl)₂, 3′,4′-(Cl)₂, 3′-Br and 4′-F, 4′-COOH, 4′-phenyl, 4′-NO₂, 2′-Br, 3′-Br, 4′-Br.

In one embodiment of the invention, wherein each of R₁, R₂ and R₃ is OH or OCH₃.

In another embodiment, wherein X is 3′-Br, 4′-Br, and R₁═R₂═OH, R₃═OH or OCH₃.

In further embodiment of the invention, wherein X is 3′-Br, 4′-Br, and R₁═R₂═OH, R₃═OCH₃.

In a further aspect, the invention provides a method for treating or preventing influenza virus infection in a subject comprising administering the subject with a pharmaceutical composition comprising a pharmaceutically acceptable carrier and a therapeutically effective amount of a compound having the general Formula I, wherein the influenza virus is H1N1 virus or H3N2 virus.

In a further yet further aspect, the invention provides a method for treating or preventing influenza virus infection in a subject comprising administering the subject with a pharmaceutical composition comprising a pharmaceutically acceptable carrier and a therapeutically effective amount of a compound having the general Formula I, wherein the influenza virus is Tamiflu-resistant influenza virus.

It is believed that a person of ordinary knowledge in the art where the present invention belongs can utilize the present invention to its broadest scope based on the descriptions herein with no need of further illustration. Therefore, the following descriptions should be understood as of demonstrative purpose instead of limitative in any way to the scope of the present invention.

### DETAILED DESCRIPTION OF THE INVENTION

Unless otherwise defined, all technical and scientific terms used herein have the same meaning as commonly understood by one of ordinary skill in the art to which this invention pertains. In the case of conflict, the present document, including definitions will be controlled.

As used herein, the singular forms “a”, “an”, and “the” include plural referents unless the context clearly dictates otherwise. Thus, for example, reference to “a sample” includes a plurality of such samples and equivalents thereof known to those skilled in the art.

As used herein, the term “subject” refers to a human or a mammal, such as a patient, a companion animal (e.g., dog, cat, and the like), a farm animal (e.g., cow, sheep, pig, horse, and the like) or a laboratory animal (e.g., rat, mouse, rabbit, and the like).

The term “therapeutically effective amount” as used herein refers to an amount of an agent sufficient to achieve the intended purpose for treatment. The therapeutically effective amount of a given agent will vary with factors such as the nature of the agent, the route of administration, the size and species of the animal to receive the agent, and the purpose of the administration. The therapeutically effective amount in each individual case may be determined empirically by a skilled artisan according to the disclosure herein and established methods in the art.

The pharmaceutical composition of the invention may be administered in any route that is appropriate, including but not limited to parenteral or oral administration. The pharmaceutical compositions for parenteral administration include solutions, suspensions, emulsions, and solid injectable compositions that are dissolved or suspended in a solvent immediately before use. The injections may be prepared by dissolving, suspending or emulsifying one or more of the active ingredients in a diluent. Examples of said diluents are distilled water for injection, physiological saline, vegetable oil, alcohol, and a combination thereof. Further, the injections may contain stabilizers, solubilizers, suspending agents, emulsifiers, soothing agents, buffers, preservatives, etc. The injections are sterilized in the final formulation step or prepared by sterile procedure.

According to the invention, the pharmaceutical composition may be in a solid or liquid form. The solid forms include tablets, pills, capsules, dispersible powders, granules, and the like. The oral pharmaceutical compositions also include gargles which are to be stuck to oral cavity and sublingual tablets. The capsules include hard capsules and soft capsules. In such solid compositions for oral use, one or more of the active compound(s) may be admixed solely or with diluents, binders, disintegrators, lubricants, stabilizers, solubilizers, and then formulated into a preparation in a conventional manner. When necessary, such preparations may be coated with a coating agent, or they may be coated with two or more coating layers. On the other hand, the liquid forms for oral administration include pharmaceutically acceptable aqueous solutions, suspensions, emulsions, syrups, elixirs, and the like. In such pharmaceutical compositions, one or more of the active compound(s) may be dissolved, suspended or emulsified in a commonly used diluent (such as purified water, ethanol or a mixture thereof, etc.). Besides such diluents, said pharmaceutical compositions may also contain wetting agents, suspending agents, emulsifiers, sweetening agents, flavoring agents, perfumes, preservatives and buffers and the like.

It is first discovered in the present invention that a novel series of synthetic compounds have effects in inhibiting influenza virus activity. It is evidenced in the present invention that new compounds provides similar or better effects than the compounds or drugs for treating or preventing influenza virus infection, by inhibiting neuraminidase (NA) activity such as baicalein, baicalin, and ribavirin. Accordingly, the present invention provides a new compound/pharmaceutical composition/method for treating or preventing influenza virus infection, particularly the Tamiflu-resistant influenza virus.

According to the present invention, it is unexpectedly found that a new compound having the structure of Formula I or a pharmaceutically acceptable salt or a physiologically functional derivative thereof:

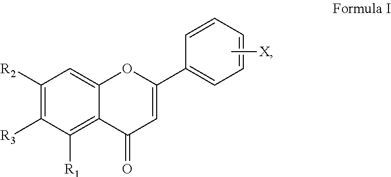

wherein X is F, Cl, Br, I, COOH, phenyl, NO₂;
each of R₁, R₂ and R₃ is OH, OCH₃ or O(CH₂)ₘ—CH₃, m is an integer of 1-3.

In one embodiment of the present invention, wherein X is 2′-Cl, 3′-Cl, 4′-Cl, 2′,4′-(Cl)₂, 3′,4′-(Cl)₂, 3′-Br and 4′-F, 4′-COOH, 4′-phenyl, 4′-NO₂, 2′-Br, 3′-Br, 4′-Br.

In another embodiment of the present invention, wherein each of R₁, R₂ and R₃ is OH or OCH₃. In one specific example, wherein X is 3′-Br, 4′-Br, and R₁═R₂═OH, R₃═OH or OCH₃.

According to the present invention, it also provided a compound, which is selected from the group consisting of:

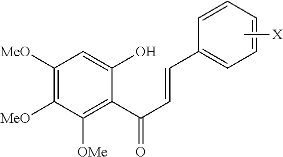

wherein X is H, 2′-Cl, 3′-Cl, 4′-Cl, 2′,4′-(Cl)₂, 3′,4′-(Cl)₂, 3′-Br and 4′-F, 4′-COOH, 4′-phenyl, 4′-NO₂, 2′-Br, 3′-Br, 4′-Br.

In one embodiment of the present invention, wherein X is 2′-Br.

According to the present invention, it provided a pharmaceutical composition for treating or preventing influenza virus infection comprising the compound of Formula I.

According to the present invention, it provided A method for treating or preventing influenza virus infection in a subject comprising administering the subject with a pharmaceutical composition comprising a pharmaceutically acceptable carrier and a therapeutically effective amount of a compound having the general Formula I:

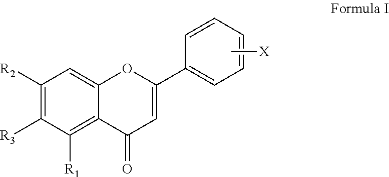

wherein X is F, Cl, Br, I, COOH, phenyl, NO₂;
each of R₁, R₂ and R₃ is OH, OCH₃ or O(CH₂)m-CH₃, m is an integer of 1-3.

In one embodiment of the present invention, wherein X is 2′-Cl, 3′-Cl, 4′-Cl, 2′,4′-(Cl)₂, 3′,4′-(Cl)₂, 3′-Br and 4′-F, 4′-COOH, 4′-phenyl, 4′-NO₂, 2′-Br, 3′-Br, 4′-Br.

In another embodiment of the present invention, wherein each of R₁, R₂ and R₃ is OH or OCH₃. In one specific example, wherein X is 3′-Br, 4′-Br, and R₁═R₂═OH, R₃═OH or OCH₃, are effective for treating or preventing influenza virus infection.

In one embodiment of the present invention, wherein the influenza virus is H1N1 virus or H3N2 virus. In one specific example, wherein the influenza virus is Tamiflu-resistant influenza virus.

The specific example below is to be construed as merely illustrative, and not limitative of the remainder of the disclosure in any way whatsoever. Without further elaboration, it is believed that one skilled in the art can, based on the description herein, utilize the present invention to its fullest extent.

### EXAMPLES

### Example 1

### Chemistry

All the chemicals were obtained from Aldrich-Sigma Chemical Company (St. Louis, Mo., USA), and Alfa-Aesar Chemical Company (Heysham, LA32XY, England) which used without further purification. All reactions were routinely monitored by TLC on Merck F254 silica gel plates. Merck silica gel (70-230 mesh) was used for chromatography. Melting points were measured on a Büchi-530 melting point apparatus. UV-VIS spectra were recorded on a Shimazu UV-160A UV-Visible recording spectrophotometer. IR spectra were registered on a Perkin-Elmer FTIR 1610 series infrared spectrophotometer in KBr discs. The ¹H-NMR and ¹³C-NMR spectra were determined on a Varian Gemini-300 NMR instrument in DMSO-d6 unless otherwise noted. Chemical shifts (δ) were reported as parts per million (ppm) downfield from tetramethylsilane (TMS) as the internal standard (σ0.00), and coupling constants (J) were given in hertz (Hz). High resolution mass (HRMS) spectra were performed in the Instrument Center of the National Science Counsel at the National Tsing-Hua University, Taiwan, using a Finnigan MAT-95XL. All the solvents and reagents were obtained from commercial sources and purified before use if necessary.

### Example 2

### Preparation of Compounds 2a-2m

The solution of cinnamic acid (10 mmol) and dichloromethane (DCM) (30 mL) was mixed in ice bath, and then a mixture of oxalyl chloride (1.5 mL, 17.5 mmol) and dimethylformamide (DMF) was added in the solution. The solution was stirred for 2 hours at room temperature, and the solvent was removed under reduced pressure to produce cinnamoyl chloride. The cinnamoyl chloride was mixed with 3,4,5-trimethoxyphenol (1.8 g, 10 mmol) and then dissolved in boron trifluoride etherate (BF₃-Et₂O)(10 mL) which was heated to reflux for 10 min. After cooling, the mixture was poured into ice water, and then the precipitate was filtered and washed with water. The precipitate was washed by hexane and then added ether to obtain the corresponding compounds 1a-1m (89-97%) in orange-yellow powders. The compounds 1a-1m could be used without further purification. A mixture of compounds 1a-m (10 mmol) and iodine (1.0 eq) in DMSO (25 mL) was heated to reflux for 3 hours. After cooling, the mixture was poured into ice water. The precipitate was filtered and washed with saturated sodium thiosulfate solution. The residue was chromatographed on a silica gel column with hexane/EtOAc (2:1) as the eluent to give pure 2a-2m in pale yellow solids.

2.1 Scheme for Compounds 2a-2m

The total synthesis of compounds 2a-2m was performed according to the scheme below:

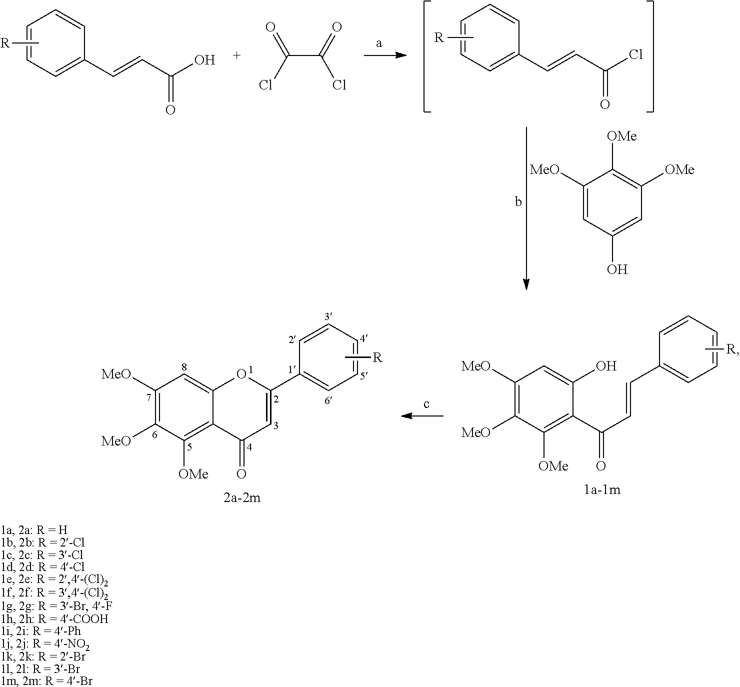

wherein the reagents and conditions are: a. DMF, DCM, room temperature; b. BF₃-Et₂O, reflux; c. I₂, DMSO, reflux.

2.2 Characterization

The spectral data of compounds 1k, 2k, 2l, and 2m were given as follows:

### (E)-3-(2-bromophenyl)-1-(6-hydroxy-2,3,4-trimethoxyphenyl)prop-2-en-1-one (1k)

¹H-NMR (300 MHz, DMSO-d₆) δ: 3.69 (3H, s, OCH₃), 3.84 (6H, s, OCH₃), 6.39 (1H, s, ArH), 7.37 (1H, td, J=7.6, 1.2 Hz, ArH), 7.47 (1H, t, J=7.6 Hz, ArH), 7.55 (1H, d, J=15.8 Hz, CH), 7.73 (1H, dd, J=9.4, 6.6 Hz, ArH), 7.83 (1H, d, J=15.8 Hz, CH), 7.91 (1H, dd, J=9.4, 6.6 Hz, ArH), 12.01 (1H, s, OH).

### 2-(2-Bromophenyl)-5,6,7-trimethoxy-4H-chromen-4-one (2k)

Yield: 61%. mp: 122-123° C. UV λₘₐₓ (MeOH) nm (log ε): 215 (4.56). IR (KBr) vcm⁻¹: 2994, 2934, 2346, 1648, 1604. HR-EI-MS m/z: 390.0110 (M⁺), Calcd for C₁₈H₁₅BrO₅: 390.0103. ¹H-NMR (300 MHz, DMSO-d₆) δ: 3.77 (3H, s, OCH₃), 3.81 (3H, s, OCH₃), 3.91 (3H, s, OCH₃), 6.34 (1H, s, ArH), 7.07 (1H, s, CHCO), 7.48-7.59 (2H, m, ArH), 7.71 (1H, dd, J=9.5, 5.7 Hz, ArH), 7.82 (1H, dd, J=9.5, 5.7 Hz, ArH). ¹³C-NMR (75 MHz, DMSO-d₆) δ: 56.4, 60.9, 61.7, 97.1, 112.1, 112.7, 121.1, 128.1, 131.2, 132.4, 133.2, 133.4, 140.1, 151.8, 154.4, 157.9, 161.2, 175.3.

### 2-(3-Bromophenyl)-5,6,7-trimethoxy-4H-chromen-4-one (2l)

Yield: 59%. mp: 190-191° C. UV λₘₐₓ (MeOH) nm (log ε): 265 (4.24), 216 (4.48). IR (KBr) vcm⁻¹: 3088, 2941, 2834, 1654, 1604. HR-EI-MS m/z: 390.0095 (M⁺), Calcd. for C₁₈H₁₅BrO₅: 390.0103. ¹H-NMR (300 MHz, DMSO-d₆) δ: 3.77 (3H, s, OCH₃), 3.80 (3H, s, OCH₃), 3.95 (3H, s, OCH₃), 6.89 (1H, s, ArH), 7.30 (1H, s, CHCO), 7.51 (1H, t, J=8.3 Hz, ArH), 7.77 (1H, dd, J=8.3 Hz, 1.2 Hz, ArH), 8.07 (1H, d, J=8.3 Hz, ArH), 8.27 (1H, t, J=2.0 Hz, ArH). ¹³C-NMR (75 MHz, DMSO-d₆) δ: 56.4, 60.8, 61.6, 97.4, 108.3, 112.1, 122.4, 125.0, 128.4, 131.0, 133.3, 134.0, 140.0, 151.5, 153.93, 157.7, 158.5, 175.5.

### 2-(4-Bromophenyl)-5,6,7-trimethoxy-4H-chromen-4-one (2m)

Yield: 67%. mp: 165-166° C. UV λₘₐₓ (MeOH) nm (log ε): 310 (4.34), 268 (3.32), 216 (4.50). IR (KBr) vcm⁻¹: 3084, 2945, 2841, 1656, 1604. HR-EI-MS m/z: 390.0110 (M⁺), Calcd for C₁₈H₁₅BrO₅: 390.0103. ¹H-NMR (300 MHz, DMSO-d₆) δ: 3.76 (3H, s, OCH₃), 3.79 (3H, s, OCH₃), 3.94 (3H, s, OCH₃), 6.86 (1H, s, ArH), 7.23 (1H, s, CHCO), 7.76 (2H, d, J=8.6 Hz, ArH), 8.01 (2H, d, J=8.6 Hz, ArH). ¹³C-NMR (75 MHz, DMSO-d₆) δ: 56.3, 60.8, 61.6, 97.2, 107.8, 112.1, 125.0, 127.9, 130.2, 132.0, 134.0, 151.6, 153.9, 157.7, 159.2, 175.6.

### Example 3

### Preparation of Compounds 3a-3j

In order to obtain the compounds 3a-3j, the compounds 2a-2j were subject to exhaustive demethylation. Thus, the compounds 2a-2j were treated with 47% HBr in acetic acid (1:2, v/v) under reflux for 48 hours followed by cooling. The crude products of 3a-3j were precipitated by addition of ice water and filtration, which were re-crystallized from ethanol to yield pure compounds.

3.1 Scheme for Compounds 3a-3j

The total synthesis of compounds 3a-3j was performed according to the scheme below:

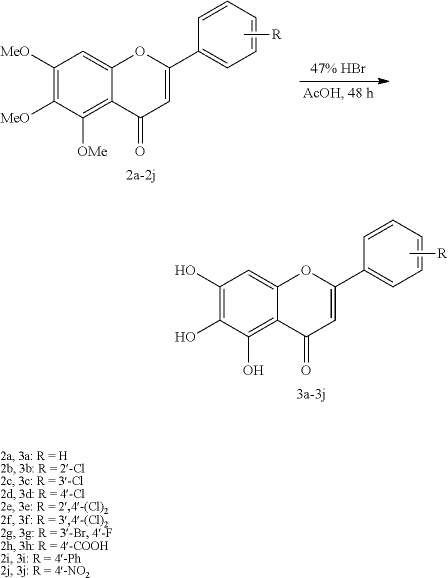

### Example 4

### Preparation of Compounds 4a-4c, 5a-5c, and 6a-6c

Compounds 2k-2m (1.0 mmol) were placed in BF₃-Et₂O (3 mL) and then heated to reflux for 45 min, respectively. After cooling, the mixture was poured into ice water. The precipitate was filtered and washed with water. The residue was chromatographed on a silica gel column with hexane/Ethyl Acetate (EtOAc) (3:1) as the eluent to give pure 4a-4c as pale yellow solids.

The solution of compounds 2k-2m (1.0 mmol) in glacial acetic acid (20 mL) was stand in the ice bath, and then the 47% hydrobromic acid (HBr) (10 mL) was added in the solution. The solution was heated to reflux for 2.5 hours. After cooling, the mixture was poured into ice water. The precipitate was filtered and washed with water. Re-crystallization from ethanol afforded pure 5a-5c in yellow solids.

The solution of compounds 2k-2m (1.0 mmol) in glacial acetic acid (20 mL) was stand in the ice bath, and then the 47% hydrobromic acid (10 mL) was added in the solution. The solution was heated to reflux for 48-54 hours. After cooling, the mixture was poured into ice water. The precipitate was filtered and washed with water. Re-crystallization from ethanol afforded pure 6a-6c in yellow-brown solids.

4.1 Scheme for Compounds 4a-4c, 5a-5c, and 6a-6c

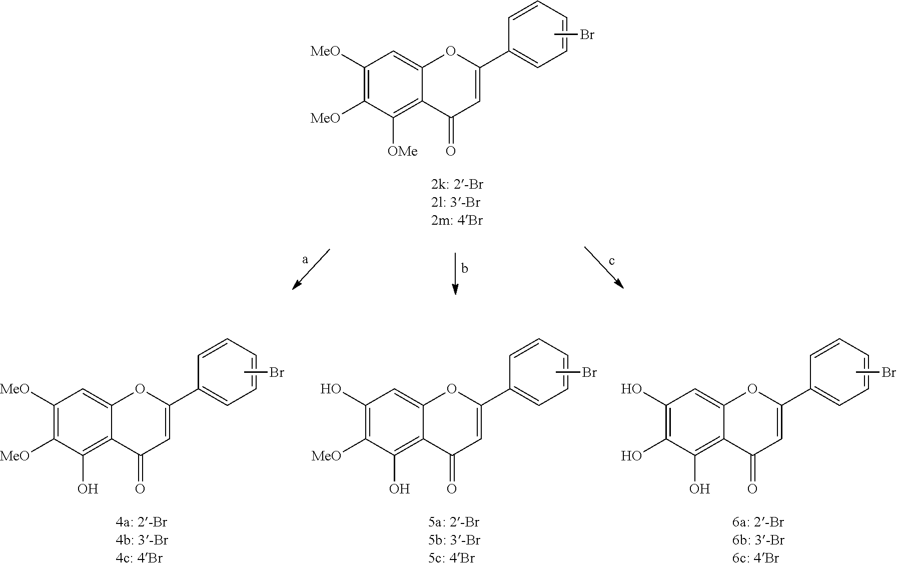

wherein the reagents and conditions are: a. BF₃-Et₂O, 45 min; b. glacial acetic acid, 47% HBr, ethanol, 2.5 h; and c. glacial acetic acid, 47% HBr, ethanol, 48-54 h.

4.2 Characterization

The spectral data of compounds 4a-4c were given as follows:

### 2-(2-Bromophenyl)-5-hydroxy-6,7-dimethoxy-4H-chromen-4-one (4a)

Yield: 69%. mp: 221-222° C. UV λₘₐₓ (MeOH) nm (log ε): 211 (4.50). IR (KBr) vcm⁻¹: 3092, 3023, 2945, 1645. HR-EI-MS m/z: 375.9946 (M⁺), Calcd for C₁₇H₁₃BrO₅: 375.9946. ¹H-NMR (300 MHz, DMSO-d₆) δ: 3.74 (3H, s, OCH₃), 3.90 (3H, s, OCH₃), 6.59 (1H, s, ArH), 6.85 (1H, s, CHCO), 7.50-7.60 (2H, m, ArH), 7.73 (1H, dd, J=9.2, 5.9 Hz, ArH), 7.84 (1H, dd, J=9.2, 5.9 Hz, ArH), 12.61 (1H, s, OH). ¹³C-NMR (75 MHz, DMSO-d₆) δ: 56.4, 59.9, 91.6, 105.3, 110.2, 120.9, 128.1, 131.4, 132.4, 132.7, 133.0, 133.5, 152.1, 153.2, 159.2, 164.4, 182.0.

### 2-(3-Bromophenyl)-5-hydroxy-6,7-dimethoxy-4H-chromen-4-one (4b)

Yield: 64%. mp: 188-189° C. UV λₘₐₓ (MeOH) nm (log ε): 272 (4.26), 215 (4.41). IR (KBr) vcm⁻¹: 3071, 2931, 2836, 1654, 1622. HR-EI-MS m/z: 375.9933 (M⁺), Calcd for C₁₇H₁₃BrO₅: 375.9946. ¹H-NMR (300 MHz, DMSO-d₆) δ: 3.73 (3H, s, OCH₃), 3.93 (3H, s, OCH₃), 7.03 (1H, s, ArH), 7.11 (1H, s, CHCO), 7.52 (1H, t, J=8.1 Hz, ArH), 7.80 (1H, dd, J=8.1 Hz, 1.5 Hz, ArH), 8.10 (1H, d, J=8.1 Hz, ArH), 8.29 (1H, t, J=1.8 Hz, ArH), 12.66 (1H, s, OH). ¹³C-NMR (75 MHz, DMSO-d₆) δ: 56.4, 59.9, 91.8, 105.4, 105.8, 122.4, 125.4, 128.8, 131.1, 132.2, 133.0, 134.6, 152.0, 152.7, 159.0, 161.7, 182.3.

### 2-(4-Bromophenyl)-5-hydroxy-6,7-dimethoxy-4H-chromen-4-one (4c)

Yield: 72%. mp: 206-208° C. UV λₘₐₓ (MeOH) nm (log ε): 278 (4.19), 215 (4.30). IR (KBr) vcm⁻¹: 2919, 2852, 1670, 1624. HR-EI-MS m/z: 375.9929 (M⁺), Calcd for C₁₇H₁₃BrO₅: 375.9946. ¹H-NMR (300 MHz, DMSO-d₆) δ: 3.73 (3H, s, OCH₃), 3.93 (3H, s, OCH₃), 6.99 (1H, s, ArH), 7.08 (1H, s, CHCO), 7.79 (2H, d, J=8.6 Hz, ArH), 8.05 (2H, d, J=8.6 Hz, ArH), 12.70 (1H, s, OH). ¹³C-NMR (75 MHz, DMSO-d₆) δ: 56.4, 59.9, 91.7, 105.3, 105.4, 125.8, 128.3, 129.6, 129.9, 132.1, 152.0, 152.8, 159.0, 162.5, 182.3.

The spectral data of compounds 5a-5c were given as follows:

### 2-(2-Bromophenyl)-5,7-dihydroxy-6-methoxy-4H-chromen-4-one (5a)

Yield: 77%. mp: 161-162° C. UV λₘₐₓ (MeOH) nm (log ε): 211 (4.54). IR (KBr) vcm⁻¹: 3092, 3066, 3005, 1680, 1622. HR-EI-MS m/z: 361.9791 (M⁺), Calcd for C₁₆H₁₁BrO₅: 361.9790. ¹H-NMR (300 MHz, DMSO-d₆) δ: 3.89 (3H, s, OCH₃), 6.53 (1H, s, ArH), 6.83 (1H, s, CHCO), 7.51-7.58 (2H, m, ArH), 7.73 (1H, dd, J=6.3, 1.8 Hz, ArH), 7.83 (1H, dd, J=5.4 Hz, 1.5 Hz, ArH), 8.83 (1H, s, OH), 12.34 (1H, s, OH). ¹³C-NMR (75 MHz, DMSO-d₆) δ: 56.3, 91.2, 105.2, 109.9, 121.0, 128.1, 130.4, 131.3, 132.6, 133.2, 133.5, 146.3, 150.2, 154.9, 164.1, 182.0.

### 2-(3-Bromophenyl)-5,7-dihydroxy-6-methoxy-4H-chromen-4-one (5b)

Yield: 72%. mp: 249-250° C. UV λₘₐₓ (MeOH) nm (log ε): 276 (4.33), 216 (4.46). IR (KBr) vcm⁻¹: 3588, 3061, 2363, 1670, 1615. HR-EI-MS m/z: 361.9789 (M⁺), Calcd for C₁₆H₁₁BrO₅: 361.9790. ¹H-NMR (300 MHz, DMSO-d₆) δ: 3.92 (3H, s, OCH₃), 7.02 (1H, s, ArH), 7.07 (1H, s, CHCO), 7.52 (1H, t, J=8.0 Hz, ArH), 7.79 (1H, dd, J=9.3, 6.9 Hz, ArH), 8.10 (1H, d, J=8.1 Hz, ArH), 8.29 (1H, t, J=1.8 Hz, ArH), 8.77 (1H, s, OH), 12.40 (1H, s, OH). ¹³C-NMR (75 MHz, DMSO-d₆) δ: 56.2, 91.4, 105.4, 105.5, 122.4, 125.3, 128.7, 130.2, 131.1, 133.2, 134.4, 146.2, 149.8, 154.8, 161.4, 182.2.

### 2-(4-Bromophenyl)-5,7-dihydroxy-6-methoxy-4H-chromen-4-one (5c)

Yield: 85%. mp: 275-276° C. UV λₘₐₓ (MeOH) nm (log ε): 282 (4.16), 218 (4.08). IR (KBr) vcm⁻¹: 3370, 3084, 2364, 2321, 1669, 1609. HR-EI-MS m/z: 361.9792 (M⁺), Calcd for C₁₆H₁₁BrO₅: 361.9790. ¹H-NMR (300 MHz, DMSO-d₆) δ: 3.91 (3H, s, OCH₃), 6.96 (1H, s, ArH), 7.04 (1H, s, CHCO), 7.79 (2H, d, J=8.9 Hz, ArH), 8.04 (2H, d, J=8.9 Hz, ArH), 8.79 (1H, s, OH), 12.70 (1H, s, OH). ¹³C-NMR (75 MHz, DMSO-d₆) δ: 56.2, 91.3, 105.0, 105.3, 125.6, 128.2, 130.2, 130.2, 132.1, 146.2, 149.8, 154.8, 162.1, 182.3.

The spectral data of compounds 6a-6c were given as follows:

### 2-(2-Bromophenyl)-5,6,7-trihydroxy-4H-chromen-4-one (6a)

Yield: 75%. mp: 249-251° C. UV λₘₐₓ (MeOH) nm (log ε): 213 (4.55). IR (KBr) vcm⁻¹: 3416, 3378, 3053, 1665, 1618. HR-EI-MS m/z: 347.9637 (M⁺), Calcd for C₁₅H₉BrO₅: 347.9633. ¹H-NMR (300 MHz, DMSO-d₆) δ: 6.46 (1H, s, ArH), 6.50 (1H, s, CHCO), 7.48-7.59 (2H, m, ArH), 7.71 (1H, dd, J=9.0, 5.7 Hz, ArH), 7.82 (1H, sd, J=8.3 Hz, ArH), 8.82 (1H, s, OH), 10.59 (1H, s, OH), 12.48 (1H, s, OH). ¹³C-NMR (75 MHz, DMSO-d₆) δ: 93.9, 104.2, 109.6, 121.0, 128.1, 129.5, 131.3, 132.6, 133.3, 133.5, 147.1, 150.2, 153.9, 163.8, 181.8.

### 2-(3-Bromophenyl)-5,6,7-trihydroxy-4H-chromen-4-one (6b)

Yield: 72%. mp: 255-256° C. UV λₘₐₓ (MeOH) nm (log ε): 276 (4.23), 211 (4.41). IR (KBr) vcm⁻¹: 3358, 3100, 2893.0, 1654, 1618. HR-EI-MS m/z: 347.9631 (M⁺), Calcd for C₁₅H₉BrO₅: 347.9633. ¹H-NMR (300 MHz, DMSO-d₆) δ: 6.65 (1H, s, ArH), 7.00 (1H, s, CHCO), 7.51 (1H, t, J=8.1 Hz, ArH), 7.78 (1H, dd, J=8.1, 1.5 Hz, ArH), 8.06 (1H, dd, J=8.1, 1.5 Hz, ArH), 8.24 (1H, t, J=1.5 Hz, ArH), 8.83 (1H, s, OH), 10.59 (1H, s, OH), 12.56 (1H, s, OH). ¹³C-NMR (75 MHz, DMSO-d₆) δ: 94.1, 104.3, 105.4, 122.4, 125.3, 128.7, 129.4, 131.1, 133.4, 134.3, 147.0, 149.9, 153.8, 161.2, 182.0.

### 2-(4-Bromophenyl)-5,6,7-trihydroxy-4H-chromen-4-one (6c)

Yield: 80%. mp: 301-303° C. UV λₘₐₓ (MeOH) nm (log ε): 265 (4.19), 216 (4.27). IR (KBr) vcm⁻¹: 3412, 3102, 1654, 1618. HR-EI-MS m/z: 347.9642 (M⁺), Calcd for C₁₅H₉BrO₅: 347.9633. ¹H-NMR (300 MHz, DMSO-d₆) δ: 6.61 (1H, s, ArH), 6.96 (1H, s, CHCO), 7.76 (2H, dd, J=7.8 Hz, 1.8 Hz, ArH), 8.01 (2H, dd, J=6.9, 2.1 Hz, ArH), 8.80 (1H, s, OH), 10.57 (1H, s, OH), 12.58 (1H, s, OH). ¹³C-NMR (75 MHz, DMSO-d₆) δ: 94.0, 104.4, 104.8, 125.5, 128.2, 129.0, 129.4, 130.3, 131.4, 132.1, 147.1, 149.9, 153.8, 161.9, 182.1.

The structures of compounds 2k-2m, 4a-4c, 5a-5c, and 6a-6c were showed in Table 1.

TABLE 1
The structures of compounds 2k-2m, 4a-4c, 5a-5c, and 6a-6c.
Formula I

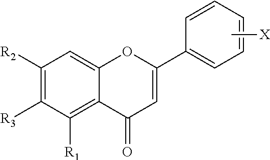

| Compound | R₁ | R₂ | R₃ | X |
|---|---|---|---|---|
| 2k | OCH₃ | OCH₃ | OCH₃ | 2′-Br |
| 2l | OCH₃ | OCH₃ | OCH₃ | 3′-Br |
| 2m | OCH₃ | OCH₃ | OCH₃ | 4′-Br |
| 4a | OH | OCH₃ | OCH₃ | 2′-Br |
| 4b | OH | OCH₃ | OCH₃ | 3′-Br |
| 4c | OH | OCH₃ | OCH₃ | 4′-Br |
| 5a | OH | OH | OCH₃ | 2′-Br |
| 5b | OH | OH | OCH₃ | 3′-Br |
| 5c | OH | OH | OCH₃ | 4′-Br |
| 6a | OH | OH | OH | 2′-Br |
| 6b | OH | OH | OH | 3′-Br |
| 6c | OH | OH | OH | 4′-Br |

The structure of compounds 3a-3j were showed in Table 2.

TABLE 2
The structures of compounds 3a-3j.
Formula I

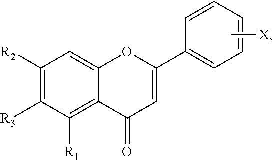

wherein R1 = R2 = R3 = OH

| Compound | X |
|---|---|
| 3a | H |
| 3b | 2′-Cl |
| 3c | 3′-Cl |
| 3d | 4′-Cl |
| 3e | 2′,4′-(Cl)₂ |
| 3f | 3′,4′-(Cl)2 |
| 3g | 3′-Br, 4′-F |
| 3h | 4′-COOH |
| 3i | 4′-Ph |
| 3j | 4′-NO₂ |

### Example 5

### In-Vitro Anti-Influenza Virus Activity

The anti-viral activity of compounds 2k-2l, 3a-3j, 4a-4c, 5a-5c, and 6a-6c was measured by the cytopathic effect (CPE) assay and used ribavirin as a positive control. The CPE inhibition assays used in this invention were performed as described in Mosmann (Mosmann T., J. Immunol. Methods, 65, 55-63, 1983). Two influenza virus strains, namely Tamiflu-resistant 2009 pandemic influenza A (H1N1) virus, which detected the H275Y mutation (N1 numbering) in neuraminidase, and influenza A/New York/469/2004-like flu (H3N2) virus, were provided by Centers for Disease Control (CDC), Taiwan, and adapted for evaluating the in vitro anti-viral activities of the compounds 2k-2l, 3a-3j, 4a-4c, 5a-5c, and 6a-6c.

In brief, virus with 100 TCID₅₀ (tissue culture infectious dose) were inoculated onto near confluent Madin-Darby canine kidney (MDCK) cell monolayers (1×10⁵ cells/well) for 1 hour. After being incubated at 37° C. for 2 hours, the virus solution was removed, and 100 μL sequential 2-fold serial dilutions of the respective compounds 2k-2l, 3a-3j, 4a-4c, 5a-5c, and 6a-6c, and the referenced compounds including Baicalein, Baicalin, and Ribavirin were added to each well of the 96-well culture plates, using the maximal non-cytotoxic concentration (MNCC, i.e. 90% viable cells) as the highest concentration. An infection control without compound was also included. The plates were incubated at 37° C. in a 75% humidity of 5% CO₂ atmosphere for 24 hours, and then the CPE was observed. The virus-induced CPE was scored as follows: scores: 0=0% CPE, 1=0-25% CPE, 2=25-50% CPE, 3=50-75% CPE, and 4=75-100% CPE. The reduction in virus multiplication was calculated as a percentage of the virus control (% virus control=CPEexp/CPEvirus control*100). The IC₅₀ of the CPE with respect to virus control was estimated using the Reed-Muench method and was expressed in μM. The selectivity index (SI) which is the value of 50% cytotoxic concentration (CC₅₀) on MDCK cells divided by the 50% effective concentration (EC₅₀) on H1N1 virus (SI=CC₅₀/EC₅₀). The higher selective index which showed the compounds were less toxic.

Results

As shown in Table 2, the most potent compounds for inhibiting influenza virus activities were compound 2l, 4b, 4c, 5b, 5c, 6b, and 6c which showed an effective anti-H1N1 Tamiflu-resistant virus activity with EC₅₀ at 4.0-4.5 μM, and the compound 4c, 5b, 5c, 6b, and 6c have the selectivity index>66.7. Compounds 2k, 4a, 5a, and 6a, however, also demonstrated an inhibitory activity (EC₅₀ at 8.6-16.0 μM) against H1N1 Tamiflu-resistant virus. The compounds with replacement of OH groups at R₁, R₂, or R₃ of Formula I can increase the inhibitory activity and diminished the cytotoxicity, especially the compounds 5b, 5c, 6b and 6c which have the bromine atom at X positions of Formula I. The presence and appropriately positioning of these hydroxyl residues at R₁, R₂, and R₃ of Formula I appeared to be critical determinants of anti-viral potency. Substitution of a bromine atom at the meta or para position of Formula I (5b, 5c, 6b, and 6c) showed the highest in vitro anti-influenza virus activity against the H1N1 Tamiflu-resistant strain and selective indexes, even superior than ribavirin.

TABLE 3
In vitro anti-H1N1 Tamiflu-resistant influenza activities of compounds
with bromo-substituted Formula I in MDCK cells using CPE assay^{a,c)}
Formula I

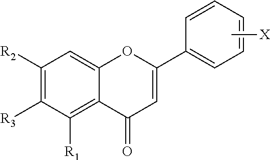

| | | | | | H1N1 Tamiflu-resistant | | |
|---|---|---|---|---|---|---|---|
| Compound | R₁ | R₂ | R₃ | X | EC₅₀ (μM) | CC₅₀ (μM) | SI |
| 2k | OCH₃ | OCH₃ | OCH₃ | 2′-Br | 16.0 | 128.2 | 8.0 |
| 2l | OCH₃ | OCH₃ | OCH₃ | 3′-Br | 4.0 | 32.1 | >8.0 |
| 2m | OCH₃ | OCH₃ | OCH₃ | 4′-Br | 192.3 | 128.2 | 0.7 |
| 4a | OH | OCH₃ | OCH₃ | 2′-Br | 16.1 | 133.0 | 8.3 |
| 4b | OH | OCH₃ | OCH₃ | 3′-Br | 4.2 | 66.5 | >15.8 |
| 4c | OH | OCH₃ | OCH₃ | 4′-Br | 4.2 | >300 | >71.4 |
| 5a | OH | OH | OCH₃ | 2′-Br | 8.6 | >300 | >34.9 |
| 5b | OH | OH | OCH₃ | 3′-Br | 4.3 | >300 | >69.8 |
| 5c | OH | OH | OCH₃ | 4′-Br | 4.3 | >300 | >70.0 |
| 6a | OH | OH | OH | 2′-Br | 9.0 | >300 | >33.3 |
| 6b | OH | OH | OH | 3′-Br | 4.5 | >300 | >66.7 |
| 6c | OH | OH | OH | 4′-Br | 4.5 | >300 | >66.7 |
| Baicalein | | | | | 46.2 | >300 | >6.5 |
| Baicalin | | | | | 69.3 | >300 | >4.3 |
| Ribavirin | | | | | 25.6 | >300 | >11.7 |

a) EC₅₀: 50% effective concentration; CC₅₀: 50% cytotoxic concentration; SI (selective index)=CC₅₀/EC₅₀. b)—: cell viability below 50-75%. c) In vitro anti-H1N1 influenza virus activity of oseltamivir (Tamiflu): EC₅₀=32.0 μM, CC₅₀=320.1 μM, SI=10; oseltamivir is void of activity against H1N1 Tamiflu-resistant virus.

Besides, the Table 4 showed that the compounds 5a and 5b have an effective anti-H3N2 virus activity. Substitution of a bromine atom at the ortho or meta position of Formula I (5a and 5b) showed the better inhibitory activity than ribavirin.

TABLE 4
In vitro anti-H3N2 influenza activities of compounds with
bromo-substituted Formula I in MDCK cells using CPE assay^{a,c)}
Formula I

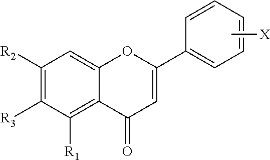

| | | | | | H3N2 | | |
|---|---|---|---|---|---|---|---|
| Compound | R₁ | R₂ | R₃ | X | EC₅₀ (μM) | CC₅₀ (μM) | SI |
| 2k | OCH₃ | OCH₃ | OCH₃ | 2′-Br | 64.1 | 128.2 | 2.0 |
| 2l | OCH₃ | OCH₃ | OCH₃ | 3′-Br | 128.2 | 64.1 | 0.5 |
| 2m | OCH₃ | OCH₃ | OCH₃ | 4′-Br | —^{b)} | >300 | — |
| 4a | OH | OCH₃ | OCH₃ | 2′-Br | 133.0 | 199.5 | 1.5 |
| 4b | OH | OCH₃ | OCH₃ | 3′-Br | 133.0 | 66.5 | 0.5 |
| 4c | OH | OCH₃ | OCH₃ | 4′-Br | — | >300 | — |
| 5a | OH | OH | OCH₃ | 2′-Br | 17.3 | >300 | >17.3 |
| 5b | OH | OH | OCH₃ | 3′-Br | 17.3 | >300 | >17.3 |
| 5c | OH | OH | OCH₃ | 4′-Br | 34.5 | >300 | >8.7 |
| 6a | OH | OH | OH | 2′-Br | 71.8 | >300 | >4.2 |
| 6b | OH | OH | OH | 3′-Br | 35.9 | >300 | >8.4 |
| 6c | OH | OH | OH | 4′-Br | 35.9 | >300 | >8.4 |
| Baicalein | | | | | 92.3 | >300 | >3.3 |
| Baicalin | | | | | 103.9 | >300 | >2.9 |
| Ribavirin | | | | | 25.6 | >300 | >11.7 |

^{a)}EC₅₀: 50% effective concentration; CC₅₀: 50% cytotoxic concentration; SI (selective index) = CC₅₀/EC₅₀.
^{b)}—: cell viability below 50-75%.
^{c)}In vitro anti-H1N1 influenza virus activity of oseltamivir (Tamiflu): EC₅₀ = 32.0 μM, CC₅₀ = 320.1 μM, SI = 10; oseltamivir is void of activity against H1N1 Tamiflu-resistant virus.

The anti-H1N1 Tamiflu-resistant influenza activities of compounds with OH group at R₁, R₂, and R₃, and different substitutions at X of Formula I were showed in Table 5. Various functionalities with different electronic effects were thus introduced and examined their anti-H1N1 Tamiflu-resistant virus activity. The compounds 3b, 3d, 3j, and 6a-6c displayed high anti-H1N1 Tamiflu-resistant influenza activities without significant cellular toxicity. However, their effective concentrations somehow appeared to distribute in a wide range. Particularly, the compounds 6a-6c which with a bromine atom attached on X position of Formula I would exhibit a significantly higher inhibitory activity (EC₅₀ at the range of 4.5-9.0 μM) than the compounds 3a-3j. It is known that the bromine atom owing to its intrinsic electronegativity can displays negative inductive effect, and among other rings would manifest a positive resonance effect after conjugation with aromatic rings by increasing the electronic density in the conjugated systems. The results distinctly revealed the significance of the impact of bromo-substitution at the X position which has the excellent anti-H1N1 Tamiflu-resistant virus activity. As the compounds 6a-6c, the chloro-substituted compounds (3b and 3d) also demonstrated highly selective index (SI>29.4). However, the compounds 6a-6c exhibited much better inhibitory activity of H1N1 Tamiflu-resistant virus than the corresponding chloro-substituent (3b-3d), especially on the 3′-position (meta position) of Formula I. The anti-H1N1 Tamiflu-resistant virus activity with para substitution on X position can be improved by the substitutions of Br, Cl, and NO₂.

TABLE 5
In vitro anti-H1N1 Tamiflu-resistant influenza virus activities of
compounds 3a-3j and 6a-6c in MDCK cells using CPE assay^{a,c)}
Formula I

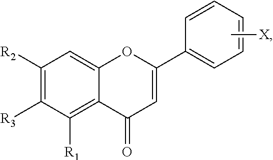

wherein R1 = R2 = R3 = OH

| | | H1N1 Tamiflu-resistant virus | | |
|---|---|---|---|---|
| Compounds | R | EC₅₀ (μM) | CC₅₀ (μM) | SI |
| 3a | H | 92.3 | >300 | >3.3 |
| 3b | 2′-Cl | 10.2 | >300 | >29.4 |
| 3c | 3′-Cl | 50.0 | >300 | >6.0 |
| 3d | 4′-Cl | 10.2 | >300 | >29.4 |
| 3e | 2′,4′-(Cl)₂ | 73.7 | >300 | >4.1 |
| 3f | 3′,4′-(Cl)₂ | —^{b)} | — | — |
| 3g | 3′-Br, 4′-F | 204.4 | >300 | >1.5 |
| 3h | 4′-COOH | 39.7 | >300 | >7.6 |
| 3i | 4′-Ph | 72.0 | >300 | >4.2 |
| 3j | 4′-NO₂ | 18.1 | >300 | >16.6 |
| 6a | 2′-Br | 9.0 | >300 | >33.3 |
| 6b | 3′-Br | 4.5 | >300 | >66.7 |
| 6c | 4′-Br | 4.5 | >300 | >66.7 |
| Baicalein | H | 92.3 | >300 | >3.3 |
| Ribavirin | | 25.6 | >300 | >11.7 |

^{a)}EC₅₀: 50% effective concentration; CC₅₀: 50% cell cytotoxic concentration; SI (selective index) = CC₅₀/EC₅₀.
^{b)}—: cell viability below 50-75%.
^{c)}In vitro anti-H1N1 influenza virus activity of oseltamivir (Tamiflu): EC₅₀ = 32.0 μM, CC₅₀ = 320.1 μM, SI = 10; oseltamivir is void of activity against H1N1 Tamiflu-resistant virus.

Statistical Analysis

All of the data in these examples were expressed as means±SEM. Statistical comparisons of the results were made using one-way analysis of variance (ANOVA). Means within each column followed by the different letters are significantly different at p<0.05 by Tukey's test.

1. A compound having the structure of Formula I or a pharmaceutically acceptable salt thereof: 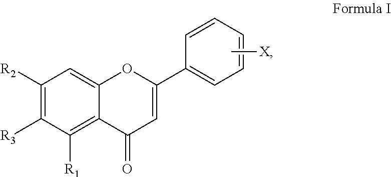
wherein X is F, Cl, 2′-Br, 3′-Br, 4′-Br, 3′-Br and 4′-F, I, COOH, phenyl, NO₂;
each of R₁, R₂ and R₃ is OH, OCH₃ or O(CH₂)ₘ—CH₃, m is an integer of 1-3.

2. The compound of claim 1, wherein X is Cl.

3. (canceled)

4. The compound of claim 1, wherein X is 2′-Cl, 3′-Cl, 4′-Cl, 2′,4′-(Cl)₂, 3′,4′-(Cl)₂, 4′-COOH, 4′-phenyl, 4′-NO₂.

5. The compound of claim 1, wherein each of R₁, R₂ and R₃ is OH or OCH₃.

6. The compound of claim 1, wherein X is 3′-Br, 4′-Br, and R₁═R₂═OH, R₃═OH or OCH₃.

7. A compound, which is selected from the group consisting of: 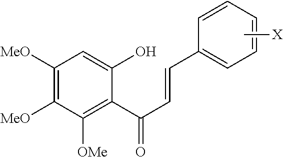
wherein X is H, 2′-Cl, 3′-Cl, 4′-Cl, 2′,4′-(Cl)₂, 3′,4′-(Cl)₂, 3′-Br and 4′-F, 4′-COOH, 4′-phenyl, 4′-NO₂, 2′-Br, 3′-Br, 4′-Br.

8. The compound of claim 7, wherein X is 2′-Br.

9. A pharmaceutical composition for treating or preventing influenza virus infection comprising the compound of claim 1.

10. A method for treating or preventing influenza virus infection in a subject comprising administering the subject with a pharmaceutical composition comprising a pharmaceutically acceptable carrier and a therapeutically effective amount of a compound having the general Formula I: 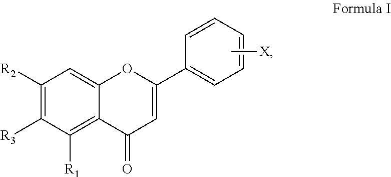
wherein X is F, Cl, Br, I, COOH, phenyl, NO₂;
each of R₁, R₂ and R₃ is OH, OCH₃ or O(CH₂)m-CH₃, m is an integer of 1-3.

11. The method of claim 10, wherein X is Cl or Br.

12. The method of claim 11, wherein X is Br.

13. The method of claim 10, wherein X is 2′-Cl, 3′-Cl, 4′-Cl, 2′,4′-(Cl)₂, 3′,4′-(Cl)₂, 3′-Br and 4′-F, 4′-COOH, 4′-phenyl, 4′-NO₂, 2′-Br, 3′-Br, 4′-Br.

14. The method of claim 10, wherein each of R₁, R₂ and R₃ is OH or OCH₃.

15. The method of claim 10, wherein X is 3′-Br, 4′-Br, and R₁═R₂═OH, R₃═OH or OCH₃.

16. The method of claim 15, wherein X is 3′-Br, 4′-Br, and R₁═R₂═OH, R₃═OCH₃.

17. The method of claim 10, wherein the influenza virus is H1N1 virus or H3N2 virus.

18. The method of claim 10, wherein the influenza virus is Tamiflu-resistant influenza virus.
